(19) United States
(12) Patent Application Publication
(10) Pub. No.: US 2024/0315530 A1
(43) Pub. Date: Sep. 26, 2024

(54) MEDICAL IMAGE PROCESSING APPARATUS AND CONTROL METHOD

(71) Applicant: Sony Olympus Medical Solutions Inc., Tokyo (JP)

(72) Inventors: Yusuke TAKENOUCHI, Tokyo (JP); Koji KOJIMA, Tokyo (JP)

(73) Assignee: Sony Olympus Medical Solutions Inc., Tokyo (JP)

(21) Appl. No.: 18/581,408

(22) Filed: Feb. 20, 2024

(30) Foreign Application Priority Data
Mar. 24, 2023 (JP) 2023-048368

(51) Int. Cl. A61B 1/00; A61B 1/045; G06T 7/00

(52) U.S. Cl. CPC A61B 1/00009; A61B 1/00059; A61B 1/045; G06T 7/00; G06T 2207/10068

(57) ABSTRACT

A medical image processing apparatus includes: a processing module configured to process image information acquired by an endoscope; and a processor configured to control an operation of the processing module. The processor is configured to change processing contents by the processing module based on a type of the connected endoscope.

### CROSS-REFERENCE TO RELATED APPLICATION

This application claims priority from Japanese Application No. 2023-048368, filed on Mar. 24, 2023, the contents of which are incorporated by reference herein in its entirety.

### BACKGROUND

The present disclosure relates to a medical image processing apparatus and a control method.

There has been known an endoscope system used in a medical field, captures an image of an inside of a living body using an imaging element, and observes the inside of the living body (see, for example, JP 2007-215850 A).

An endoscope system disclosed in JP 2007-215850 A includes an endoscope that is inserted into a living body and includes an imaging unit that images an inside of the living body, and a medical image processing apparatus that is detachably connected to the endoscope and includes a processing module that processes a captured image captured by the endoscope.

### SUMMARY

Meanwhile, various endoscopes of different release generations may be connected to one medical image processing apparatus. Then, in the various endoscopes, an imaging unit and the like are different. Therefore, in the medical image processing apparatus, there is a need to cause the processing module to perform processing with processing contents corresponding to the connected endoscope (imaging unit or the like).

For example, in the medical image processing apparatus, information specific to the connected endoscope and information indicating processing contents of the processing module corresponding to the endoscope are stored in association with each other. This configuration makes it possible to cope with a case where an existing endoscope for a medical image processing apparatus is connected to the medical image processing apparatus. However, in a case where a later endoscope for a medical image processing apparatus is connected to the medical image processing apparatus, information corresponding to the later endoscope is not stored in a memory. Therefore, it is not possible to cope with a case where a later endoscope is connected to the medical image processing apparatus. It is conceivable that information corresponding to the later endoscope is stored in the memory of the medical image processing apparatus every time the later endoscope is released, but such a measure results in poor convenience.

Therefore, there is a demand for a technique that may cope with both a case where an existing endoscope is connected and a case where a later endoscope is connected, and may improve convenience.

According to one aspect of the present disclosure, there is provided a medical image processing apparatus including: a processing module configured to process image information acquired by an endoscope; and a processor configured to control an operation of the processing module, wherein the processor is configured to change processing contents by the processing module based on a type of the connected endoscope.

According to another aspect of the present disclosure, there is provided a control method executed by a processor of a medical image processing apparatus, the control method including: causing the processor to change a processing content by a processing module configured to process image information acquired by an endoscope based on a type of the endoscope connected to the medical image processing apparatus.

### BRIEF DESCRIPTION OF THE DRAWINGS

FIG. 1 is a diagram illustrating a configuration of an endoscope system according to an embodiment;

FIG. 2 is a block diagram illustrating a configuration of a control device;

FIG. 3 is a flowchart illustrating an operation of the control device;

FIG. 4 is a diagram illustrating a first modification of the embodiment;

FIG. 5 is a diagram illustrating the first modification of the embodiment;

FIG. 6 is a diagram illustrating a second modification of the embodiment;

FIG. 7 is a diagram illustrating the second modification of the embodiment;

FIG. 8 is a diagram illustrating a third modification of the embodiment;

FIG. 9 is a diagram illustrating the third modification of the embodiment;

FIG. 10 is a diagram illustrating a fourth modification of the embodiment;

FIG. 11 is a diagram illustrating the fourth modification of the embodiment;

FIG. 12 is a diagram illustrating a fifth modification of the embodiment;

FIG. 13 is a diagram illustrating the fifth modification of the embodiment; and

FIG. 14 is a diagram illustrating a sixth modification of the embodiment.

### DETAILED DESCRIPTION

Hereinafter, modes for carrying out the present disclosure (hereinafter, embodiments) will be described with reference to the drawings. Note that the present disclosure is not limited by the embodiments described below. Furthermore, in the description of the drawings, the same portions are denoted by the same reference numerals.

### Schematic Configuration of Endoscope System

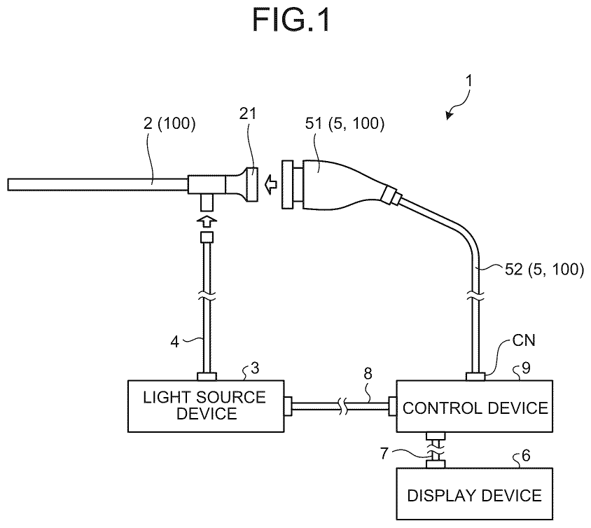

FIG. 1 is a diagram illustrating a configuration of an endoscope system 1 according to an embodiment.

The endoscope system 1 is a system that is used in a medical field and observes an inside of a living body. As illustrated in FIG. 1, the endoscope system 1 includes an insertion unit 2, a light source device 3, a light guide 4, a camera head 5, a display device 6, a second transmission cable 7, a third transmission cable 8, and a control device 9.

In the present embodiment, the insertion unit 2 includes a rigid endoscope. That is, the insertion unit 2 has an elongated shape that is entirely rigid or soft in a part and rigid in the other part, and is inserted into the living body. An optical system (not illustrated) configured using one or a plurality of lenses and configured to condense a subject image is provided in the insertion unit 2.

The light source device 3 is connected to one end of the light guide 4, and supplies an illumination light designated by the control device 9 to the one end of the light guide 4 with a light amount designated by the control device 9 under the control of the control device 9.

In the present embodiment, the light source device 3 is configured separately from the control device 9, but the present disclosure is not limited thereto, and a configuration provided in the same casing as the control device 9 may be adopted.

One end of the light guide 4 is detachably connected to the light source device 3, and the other end is detachably connected to the insertion unit 2. Then, the light guide 4 transmits the light supplied from the light source device 3 from one end to the other end, and supplies the light to the insertion unit 2. The light supplied to the insertion unit 2 is emitted from a distal end of the insertion unit 2 and emitted into the living body. The light (subject image) emitted into the living body, and returned from the living body is condensed by the optical system in the insertion unit 2.

As illustrated in FIG. 1, the camera head 5 includes a camera head body 51 detachably connected to an eyepiece unit 21 (FIG. 1) in the insertion unit 2, and a first transmission cable 52 extending from the camera head body 51 and detachably connected to the control device 9 through a connector CN provided at one end. Then, under the control of the control device 9, the camera head 5 captures the subject image condensed by the insertion unit 2 and generates an image signal (hereinafter, referred to as a captured image).

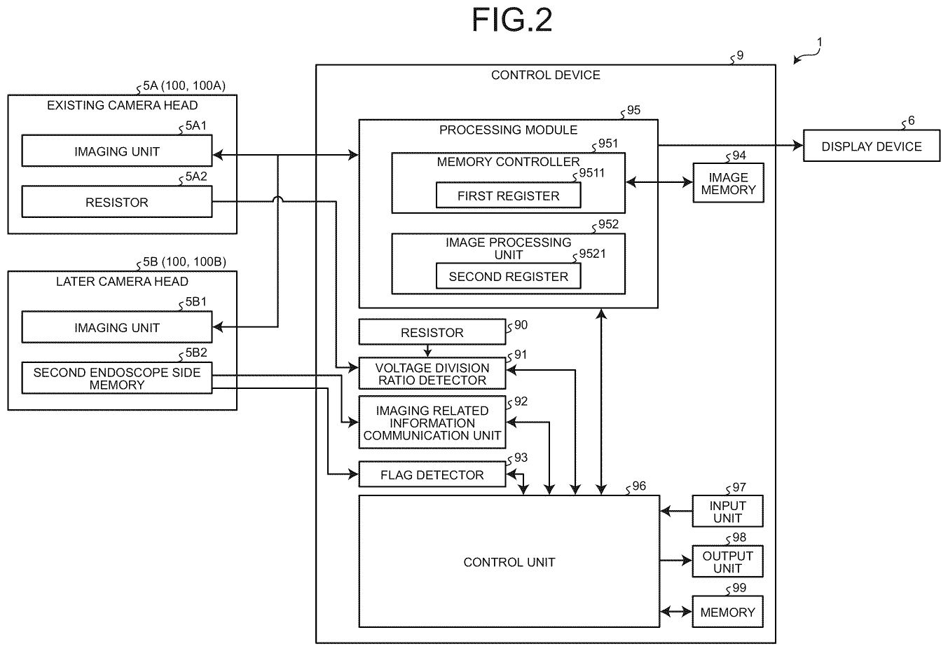

In the present embodiment, various camera heads 5 of different release generations may be connected to the control device 9. Hereinafter, the existing camera head 5 for the control device 9 is referred to as an existing camera head 5A (see FIG. 2). The later camera head 5 for the control device 9 is referred to as a later camera head 5B (see FIG. 2). Then, the insertion unit 2 and the camera head 5 correspond to an endoscope 100 (FIG. 1) according to the present disclosure. Furthermore, the insertion unit 2 and the existing camera head 5A correspond to a first endoscope 100A (see FIG. 2) according to the present disclosure. Furthermore, the insertion unit 2 and the later camera head 5B correspond to a second endoscope 100B (see FIG. 2) according to the present disclosure.

One or a plurality of existing camera heads 5A may be provided as long as the existing camera heads 5 are provided for the control device 9. The existing camera head 5A includes an imaging unit 5A1 (see FIG. 2) and a resistor 5A2 (see FIG. 2).

The imaging unit 5A1 includes an imaging element such as a charge coupled device (CCD) or a complementary metal oxide semiconductor (CMOS) that receives the subject image condensed by the insertion unit 2 and converts the subject image into an electric signal. Then, under the control of the control device 9, the imaging unit 5A1 captures the subject image condensed by the insertion unit 2 to generate a captured image. The captured image corresponds to image information according to the present disclosure.

The resistor 5A2 corresponds to a first resistor according to the present disclosure. The resistor 5A2 is a resistor having a resistance value unique to the existing camera head 5A.

One or a plurality of later camera heads 5B may be provided as long as the later camera heads 5 are provided for the control device 9. The later camera head 5B includes an imaging unit 5B1 (see FIG. 2) and a second endoscope side memory 5B2 (see FIG. 2).

The imaging unit 5B1 includes an imaging element such as a CCD or a CMOS that receives the subject image condensed by the insertion unit 2 and converts the subject image into an electric signal. Then, under the control of the control device 9, the imaging unit 5B1 captures the subject image condensed by the insertion unit 2 to generate a captured image. The captured image corresponds to image information according to the present disclosure.

The second endoscope side memory 5B2 stores imaging related information and a later scope flag.

The imaging related information corresponds to second setting information according to the present disclosure. The imaging related information is information unique to the later camera head 5B, and is information related to processing contents of the captured image generated by the imaging unit 5B1 in the later camera head 5B by a processing module 95 (see FIG. 2) to be described later. Specifically, the imaging related information includes an image format related to the imaging unit 5B1 and image processing parameters.

The image format includes an image size of the captured image generated by the imaging unit 5B1 and sizes of writing and reading to and from an image memory 94 (see FIG. 2) to be described later.

The image processing parameter is an image processing parameter for the processing module 95 to perform image processing on the captured image generated by the imaging unit 5B1.

The later scope flag corresponds to second identification information according to the present disclosure. The later scope flag is set to ON when the camera head 5 is the later camera head 5B.

Here, the first transmission cable 52 transmits a captured image and the like output from the camera head body 51 (imaging units 5A1 and 5B1) to the control device 9, and transmits a control signal, a synchronization signal, a clock, power, and the like output from the control device 9 to the camera head body 51.

Note that, in the transmission of the captured image and the like from the camera head body 51 to the control device 9 through the first transmission cable 52, the captured image and the like may be transmitted as an optical signal or may be transmitted as an electric signal. The same applies to transmission of a control signal, a synchronization signal, and a clock from the control device 9 to the camera head body 51 through the first transmission cable 52.

The display device 6 includes a display using liquid crystal, organic electro luminescence (EL), or the like, and displays an image based on a video signal from the control device 9 under the control of the control device 9.

One end of the second transmission cable 7 is detachably connected to the display device 6, and the other end is detachably connected to the control device 9. Then, the second transmission cable 7 transmits the video signal processed by the control device 9 to the display device 6.

One end of the third transmission cable 8 is detachably connected to the light source device 3, and the other end is detachably connected to the control device 9. Then, the third transmission cable 8 transmits the control signal from the control device 9 to the light source device 3.

The control device 9 corresponds to a medical image processing apparatus according to the present disclosure. The control device 9 includes a central processing unit (CPU), a field-programmable gate array (FPGA), and the like, and integrally controls operations of the light source device 3, the camera head 5, and the display device 6.

Note that a detailed configuration of the control device 9 will be described in “Configuration of control device” described later.

### Configuration of Control Device

Next, a configuration of the control device 9 will be described.

FIG. 2 is a block diagram illustrating a configuration of a control device 9.

As illustrated in FIG. 2, the control device 9 includes a resistor 90, a voltage division ratio detector 91, an imaging related information communication unit 92, a flag detector 93, an image memory 94, a processing module 95, a control unit 96, an input unit 97, an output unit 98, and a storage unit 99.

The resistor 90 corresponds to a second resistor according to the present disclosure. The resistor 90 is a resistor having a resistance value unique to the control device 9.

The voltage division ratio detector 91 detects a voltage division ratio based on a resistance value of the resistor 90 and a resistance value of the resistor 5A2 in the existing camera head 5A under the control of the control unit 96.

The imaging related information communication unit 92 acquires the imaging related information stored in the second endoscope side memory 5B2 in the later camera head 5B under the control of the control unit 96.

The flag detector 93 checks the later scope flag stored in the camera head 5 under the control of the control unit 96.

The image memory 94 includes, for example, a dynamic random access memory (DRAM) or the like. The image memory 94 may temporarily store a plurality of frames of captured images sequentially output from the camera head 5.

The processing module 95 includes, for example, a field-programmable gate array (FPGA), and processes the captured images sequentially output from the camera head 5. As illustrated in FIG. 2, the processing module 95 includes a memory controller 951 and an image processing unit 952.

The memory controller 951 includes a first register 9511 that stores an image format, and controls writing of a captured image to the image memory 94 and reading from the image memory 94 according to the image format stored in the first register 9511. Specifically, the memory controller 951 sequentially writes the captured images sequentially output from the camera head 5 in the image memory 94 with a write size corresponding to the image format. In addition, the memory controller 951 reads the captured image from the image memory 94 with a reading size corresponding to the image format, and inputs the read captured image to the image processing unit 952.

The image processing unit 952 includes a second register 9521 that stores image processing parameters, and uses the image processing parameters stored in the second register 9521 to perform image processing on the captured image read from the image memory 94 by the memory controller 951. In addition, the image processing unit 952 generates a display image (a video signal for display) for displaying the captured image after undergoing the image processing. Then, the image processing unit 952 outputs the display image to the display device 6. As a result, the display image is displayed on the display device 6.

Specifically, examples of the image processing include optical black subtraction processing, demosaic processing, white balance adjustment processing, digital gain processing (processing of multiplying a digital signal by a digital gain for amplifying the digital signal), noise reduction processing, color correction processing, color enhancement processing, contour enhancement processing, enlargement processing, color tone change processing, YC processing of converting an RGB signal (captured image) into a luminance color difference signal (Y, Cb/Cr SIGNAL), and the like.

The control unit 96 corresponds to a processor according to the present disclosure. The control unit 96 is realized by executing various programs stored in the storage unit 99 by a controller such as a central processing unit (CPU) or a micro processing unit (MPU), and controls the operations of the light source device 3, the camera head 5, and the display device 6 and controls the entire operation of the control device 9. Note that the control unit 96 is not limited to the CPU or the MPU, and may be configured by an integrated circuit such as an application specific integrated circuit (ASIC) or an FPGA. Note that the function of the control unit 96 will be described in “Operation of control device” described later.

The input unit 97 is configured using an operation device such as a mouse, a keyboard, and a touch panel, and receives a user operation by a user such as an operator. Then, the input unit 97 outputs an operation signal corresponding to the user operation to the control unit 96.

The output unit 98 is configured using a speaker, a printer, or the like, and outputs various types of information.

The storage unit 99 corresponds to a memory according to the present disclosure. The storage unit 99 stores a program executed by the control unit 96, information necessary for processing of the control unit 96, and the like. As the information necessary for the processing of the control unit 96, a correspondence table may be exemplified.

The correspondence table is a table in which each voltage division ratio based on the resistance value of the resistor 90 of the control device 9 and each resistance value of each resistor 5A2 according to the plurality of existing camera heads 5A is associated with the first setting information for each of the plurality of existing camera heads 5A.

Here, the first setting information is information unique to the existing camera head 5A, and is information regarding processing contents of the captured image generated by the imaging unit 5A1 in the existing camera head 5A by the processing module 95. Specifically, the first setting information includes an image format and an image processing parameter related to the imaging unit 5A1.

The image format includes the image size of the captured image generated by the imaging unit 5A1 and the size of writing and reading to and from the image memory 94.

The image processing parameter is an image processing parameter for the processing module 95 to perform image processing on the captured image generated by the imaging unit 5A1.

### Operation of Control Device

Next, an operation of the control device 9 (corresponding to a control method according to the present disclosure) will be described.

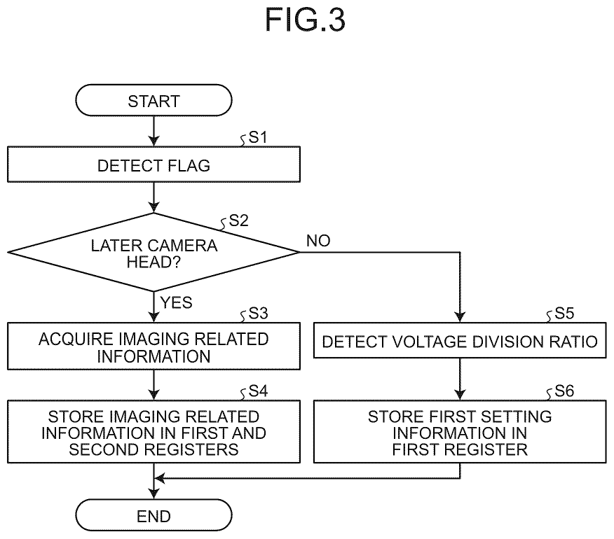

FIG. 3 is a flowchart illustrating the operation of the control device 9.

First, the flag detector 93 detects the later scope flag stored in the camera head 5 under the control of the control unit 96 (Step S1). Then, the flag detector 93 outputs a signal indicating the detection result to the control unit 96.

Then, in Step S1, in a case where the later scope flag may not be confirmed by the flag detector 93, the control unit 96 determines that the camera head 5 connected to the control device 9 is the existing camera head 5A (Step S2: No). On the other hand, in Step S1, when the flag detector 93 detects that the later scope flag is set to ON, the control unit 96 determines that the camera head 5 connected to the control device 9 is the later camera head 5B (Step S2: Yes).

In a case where it is determined that the camera head 5 connected to the control device 9 is the later camera head 5B (Step S2: Yes), the imaging related information communication unit 92 acquires the imaging related information stored in the second endoscope side memory 5B2 in the later camera head 5B under the control of the control unit 96 (Step S3).

After Step S3, the control unit 96 stores the image format included in the imaging related information acquired in Step S3 in the first register 9511, and stores the image processing parameter included in the imaging related information in the second register 9521 (Step S4).

As a result, the memory controller 951 controls writing of the captured image to the image memory 94 and reading from the image memory 94 according to the image format included in the imaging related information. In addition, the image processing unit 952 performs image processing on the captured image read from the image memory 94 by the memory controller 951 using the image processing parameter included in the imaging related information.

On the other hand, in a case where it is determined that the camera head 5 connected to the control device 9 is the existing camera head 5A (Step S2: No), the voltage division ratio detector 91 detects the voltage division ratio based on the resistance value of the resistor 90 and the resistance value of the resistor 5A2 in the existing camera head 5A under the control of the control unit 96 (Step S5).

After Step S5, the control unit 96 refers to the correspondence table stored in the storage unit 99 and recognizes the first setting information associated with the voltage division ratio detected in Step S5. Then, the control unit 96 stores the image format included in the first setting information in the first register 9511, and stores the image processing parameter included in the first setting information in the first register 9511 (Step S6).

As a result, the memory controller 951 controls writing of the captured image into the image memory 94 and reading from the image memory 94 according to the image format included in the first setting information. In addition, the image processing unit 952 uses the image processing parameter included in the first setting information to perform image processing on the captured image read from the image memory 94 by the memory controller 951.

As described above, the control unit 96 changes the processing contents by the processing module 95 based on the type of the endoscope 100 connected to the control device 9. In addition, the control unit 96 changes the processing contents by the processing module 95 to different processing contents depending on whether the endoscope 100 connected to the control device 9 is the first endoscope 100A corresponding to the first setting information stored in the storage unit 99 or the second endoscope 100B not corresponding to the first setting information. Furthermore, in a case where the endoscope 100 connected to the control device 9 is the first endoscope 100A, the control unit 96 causes the processing module 95 to execute processing based on the first setting information. On the other hand, in a case where the endoscope 100 connected to the control device 9 is the second endoscope 100B, the control unit 96 causes the processing module 95 to execute processing based on the imaging related information.

According to the present embodiment described above, the following effects are obtained.

In the control device 9 according to the present embodiment, the control unit 96 changes the processing contents by the processing module 95 based on the type of the endoscope 100 connected to the control device 9. Specifically, the control unit 96 determines the type of the endoscope 100 based on the later scope flag stored in the later camera head 5B. Then, in a case where the camera head 5 connected to the control device 9 is determined to be the later camera head 5B, the control unit 96 stores the imaging related information acquired from the later camera head 5B in each of the first and second registers 9511 and 9521. On the other hand, in a case where the camera head 5 connected to the control device 9 is determined to be the existing camera head 5A, the control unit 96 refers to the setting table stored in the storage unit 99, and causes the first and second registers 9511 and 9521 to store the first setting information associated with the voltage division ratio based on the resistance value of the resistor 90 and the resistance value of the resistor 5A2 in the existing camera head 5A.

Therefore, according to the control device 9 according to the present embodiment, the processing contents by the processing module 95 may be changed both in a case where the existing camera head 5A is connected and in a case where the later camera head 5B is connected, and convenience may be improved.

### Other Embodiments

Although the embodiments for carrying out the present disclosure have been described so far, the present disclosure should not be limited only by the above-described embodiments.

Configurations of first to sixth modifications described below may be adopted.

### First Modification

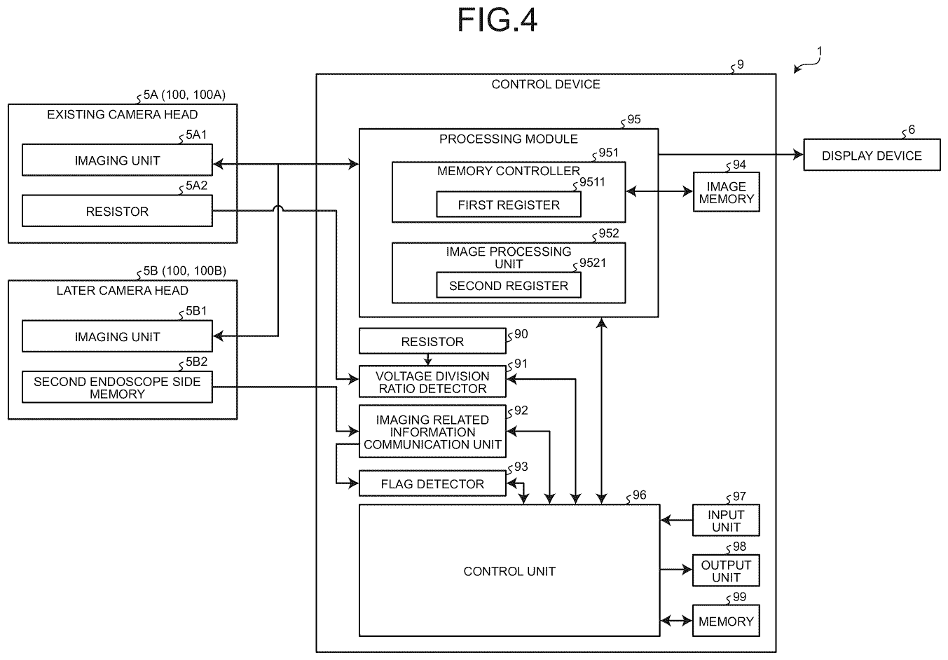

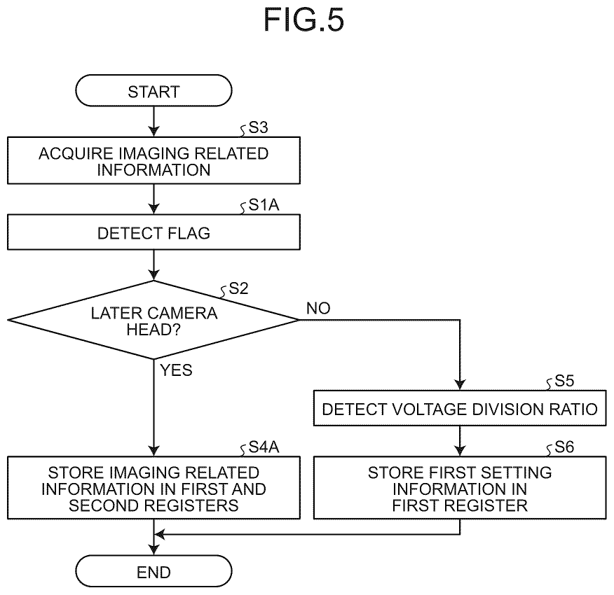

FIGS. 4 and 5 are diagrams illustrating a first modification of the embodiment. Specifically, FIG. 4 is a block diagram corresponding to FIG. 2. FIG. 5 is a flowchart corresponding to FIG. 3.

In the above-described embodiment, the later scope flag is stored in the second endoscope side memory 5B2 independently of the imaging related information. However, the present disclosure is not limited thereto, and the later scope flag may be included in the imaging related information.

In such a configuration, as illustrated in FIG. 4, the flag detector 93 detects the later scope flag included in the imaging related information acquired by the imaging related information communication unit 92.

In the first modification, the control device 9 operates as described below.

First, the imaging related information communication unit 92 acquires the imaging related information stored in the second endoscope side memory 5B2 in the later camera head 5B under the control of the control unit 96 (Step S3).

After Step S3, the flag detector 93 detects the later scope flag included in the imaging related information acquired in Step S3 under the control of the control unit 96 (Step S1A). Then, the flag detector 93 outputs a signal indicating the detection result to the control unit 96.

Then, in a case where the later scope flag may not be confirmed by the flag detector 93 in Step S1A, the control unit 96 determines that the camera head 5 connected to the control device 9 is the existing camera head 5A (Step S2: No). On the other hand, in Step S1A, when the flag detector 93 detects that the later scope flag is set to ON, the control unit 96 determines that the camera head 5 connected to the control device 9 is the later camera head 5B (Step S2: Yes).

When it is determined that the camera head 5 connected to the control device 9 is the later camera head 5B (Step S2: Yes), the control unit 96 stores the image format included in the imaging related information acquired in Step S3 in the first register 9511, and stores the image processing parameter included in the imaging related information in the second register 9521 (Step S4A).

On the other hand, in a case where it is determined that the camera head 5 connected to the control device 9 is the existing camera head 5A (Step S2: No), the control device 9 executes the processing of Steps S5 and S6 similarly to the above-described embodiment.

Even in a case where the configuration of the first modification described above is adopted, the same effects as those of the above-described embodiment are obtained.

### Second Modification

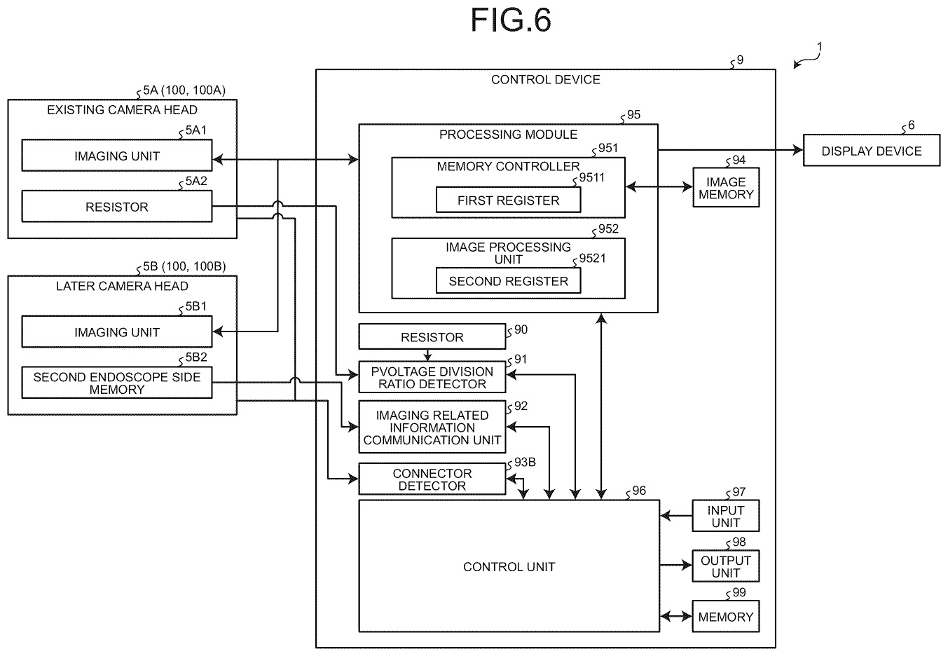

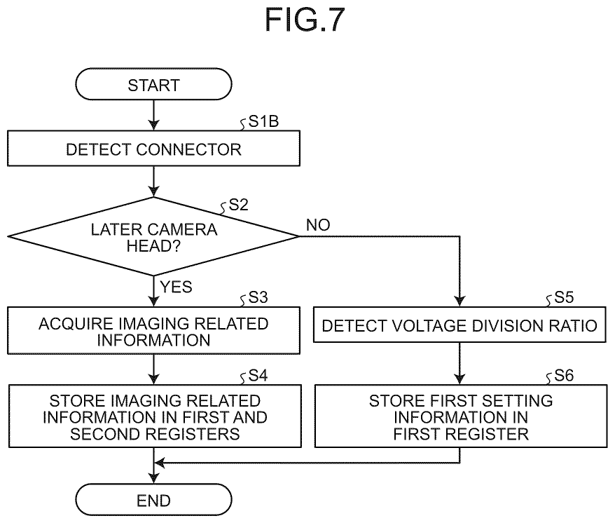

FIGS. 6 and 7 are diagrams illustrating a second modification of the embodiment. Specifically, FIG. 6 is a block diagram corresponding to FIG. 2. FIG. 7 is a flowchart corresponding to FIG. 3.

In the above-described embodiment, instead of the flag detector 93, a connector detector 93B may be adopted as in the present second modification illustrated in FIG. 6.

Here, the connector CN in the later camera head 5B according to the second modification is different from the connector CN in the existing camera head 5A in connector shape and pin arrangement.

The connector detector 93B detects a connector shape and pin arrangement in the connector CN of the camera head 5 connected to the control device 9 under the control of the control unit 96. Then, the connector detector 93B outputs a signal indicating the detection result to the control unit 96.

In the second modification, the control device 9 operates as described below.

First, the connector detector 93B detects a connector shape and pin arrangement in the connector CN of the camera head 5 connected to the control device 9 under the control of the control unit 96 (Step S1B). Then, the connector detector 93B outputs a signal indicating the detection result to the control unit 96.

Then, the control unit 96 determines whether the camera head 5 connected to the control device 9 is the existing camera head 5A (Step S2: No) or the later camera head 5B (Step S2: Yes) based on the connector shape and the pin arrangement in the connector CN detected by the connector detector 93B in Step S1B.

Even in a case where the configuration of the second modification described above is adopted, the same effects as those of the above-described embodiment are obtained.

### Third Modification

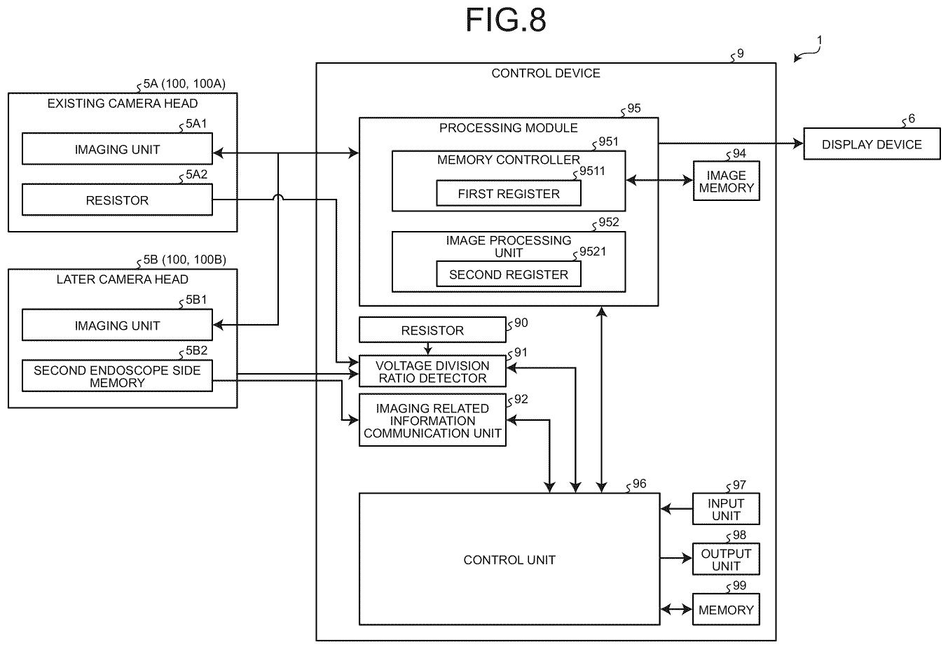

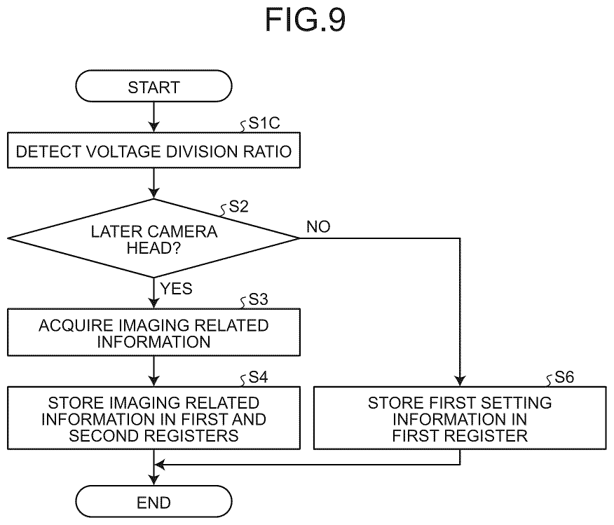

FIGS. 8 and 9 are diagrams illustrating a third modification of the embodiment. Specifically, FIG. 8 is a block diagram corresponding to FIG. 2. FIG. 9 is a flowchart corresponding to FIG. 3.

In the above-described embodiment, as in the present third modification illustrated in FIG. 8, the function of the flag detector 93 may be substituted by the voltage division ratio detector 91, and the flag detector 93 may not be provided.

In the third modification, the control device 9 operates as described below.

First, the voltage division ratio detector 91 detects a voltage division ratio based on the resistance value of the resistor 90 and the resistance value of the resistor in the camera head 5 connected to the control device 9 under the control of the control unit 96 (Step S1C). Then, the voltage division ratio detector 91 outputs a signal indicating the detection result to the control unit 96.

Then, the control unit 96 refers to the correspondence table stored in the storage unit 99, and determines whether or not the voltage division ratio detected by the voltage division ratio detector 91 in Step S1C has been registered. Here, when determining that the voltage division ratio detected by the voltage division ratio detector 91 has been registered in the correspondence table, the control unit 96 determines that the camera head 5 connected to the control device 9 is the existing camera head 5A (Step S2: No). On the other hand, when determining that the voltage division ratio detected by the voltage division ratio detector 91 has not been registered in the correspondence table, the control unit 96 determines that the camera head 5 connected to the control device 9 is the later camera head 5B (Step S2: Yes).

In the third modification, since the voltage division ratio has already been detected in Step S1C, Step S5 is not performed.

Even in a case where the configuration of the present third modification described above is adopted, the same effects as those of the above-described embodiment are obtained.

### Fourth Modification

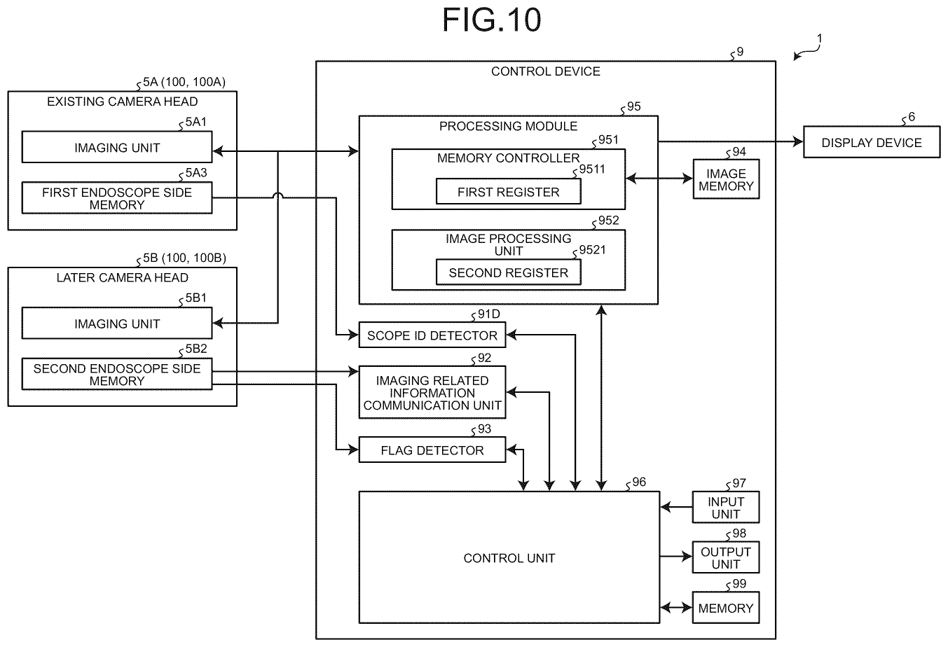

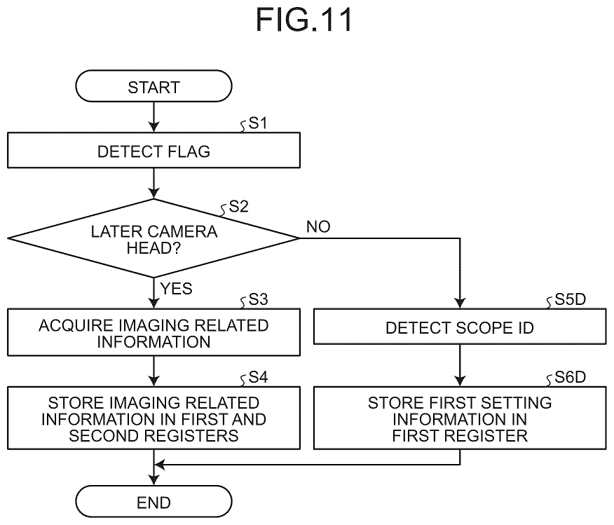

FIGS. 10 and 11 are diagrams illustrating a fourth modification of the embodiment. Specifically, FIG. 10 is a block diagram corresponding to FIG. 2. FIG. 11 is a flowchart corresponding to FIG. 3.

In the above-described embodiment, instead of the voltage division ratio detector 91, a scope ID detector 91D may be adopted as in the present fourth modification illustrated in FIG. 10.

Here, as illustrated in FIG. 10, the existing camera head 5A according to fourth modification is provided with a first endoscope side memory 5A3 that stores a scope ID unique to the existing camera head 5A instead of the resistor 5A2. The scope ID corresponds to first identification information according to the present disclosure.

The scope ID detector 91D acquires the scope ID stored in the first endoscope side memory 5A3 in the existing camera head 5A under the control of the control unit 96.

Furthermore, the correspondence table stored in the storage unit 99 according to the present fourth modification is a table in which each scope ID corresponding to the plurality of existing camera heads 5A is associated with the first setting information for each of the plurality of existing camera heads 5A.

In the fourth modification, the control device 9 operates as described below.

In the operation of the control device 9 according to the fourth modification, as illustrated in FIG. 11, Steps S5D and S6D are adopted instead of Steps S5 and S6 for the operation of the control device 9 described in the above-described embodiment.

Specifically, in Step S5D, the scope ID detector 91D acquires the scope ID stored in the first endoscope side memory 5A3 in the existing camera head 5A under the control of the control unit 96.

After Step S5D, the control unit 96 refers to the correspondence table stored in the storage unit 99 and recognizes the first setting information associated with the scope ID acquired in Step S5D. Then, the control unit 96 stores the image format included in the first setting information in the first register 9511, and stores the image processing parameter included in the first setting information in the first register 9511 (Step S6D).

Even in a case where the configuration of the fourth modification described above is adopted, the same effects as those of the above-described embodiment are obtained.

### Fifth Modification

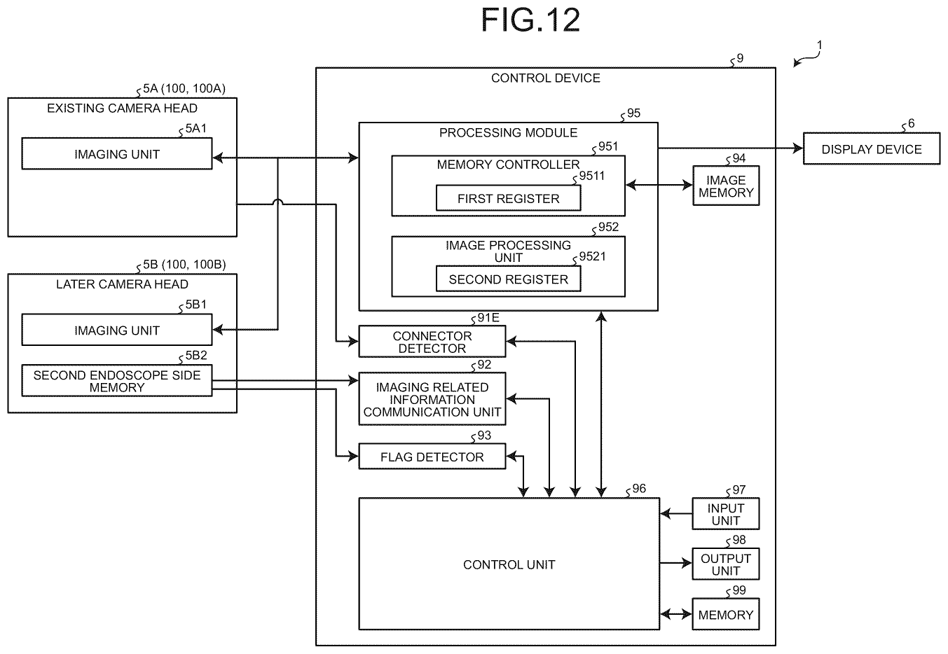

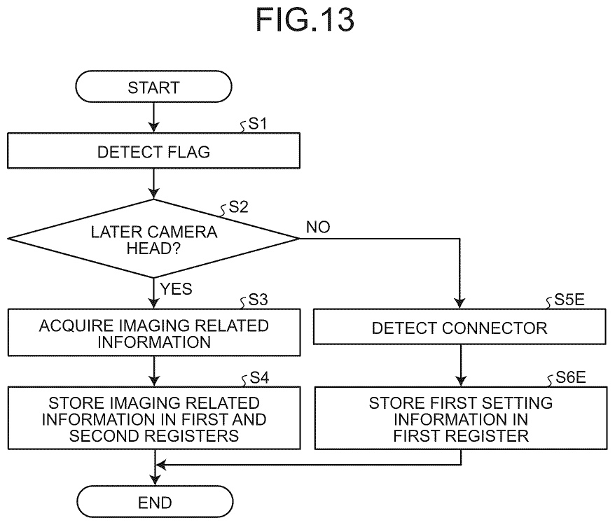

FIGS. 12 and 13 are diagrams illustrating a fifth modification of the embodiment. Specifically, FIG. 12 is a block diagram corresponding to FIG. 2. FIG. 13 is a flowchart corresponding to FIG. 3.

In the above-described embodiment, instead of the voltage division ratio detector 91, a connector detector 91E may be adopted as in the present fifth modification illustrated in FIG. 12.

Here, the connector CN in the existing camera head 5A has a unique connector shape and pin arrangement.

The connector detector 91E detects a connector shape and pin arrangement in the connector CN of the existing camera head 5A connected to the control device 9 under the control of the control unit 96. Then, the connector detector 91E outputs connector information indicating a connector shape and pin arrangement in the detected connector CN to the control unit 96.

Furthermore, the correspondence table stored in the storage unit 99 according to the present fifth modification is a table in which each connector information corresponding to the plurality of existing camera heads 5A is associated with the first setting information for each of the plurality of existing camera heads 5A.

In the fifth modification, the control device 9 operates as described below.

In the operation of the control device 9 according to the fifth modification, as illustrated in FIG. 13, Steps S5E and S6E are adopted instead of Steps S5 and S6 for the operation of the control device 9 described in the above-described embodiment.

Specifically, in Step S5E, the connector detector 91E detects the connector shape and the pin arrangement in the connector CN of the existing camera head 5A connected to the control device 9 under the control of the control unit 96. Then, the connector detector 91E outputs connector information indicating a connector shape and pin arrangement in the detected connector CN to the control unit 96.

After Step S5E, the control unit 96 refers to the correspondence table stored in the storage unit 99, and recognizes the first setting information associated with the connector information output from the connector detector 91E in Step S5E. Then, the control unit 96 stores the image format included in the first setting information in the first register 9511, and stores the image processing parameter included in the first setting information in the first register 9511 (Step S6E).

Even in a case where the configuration of the fifth modification described above is adopted, the same effects as those of the above-described embodiment are obtained.

### Sixth Modification

An endoscope system according to a sixth modification is an endoscope system using a so-called video scope (flexible endoscope) having an imaging unit on a distal end side of an insertion unit. Hereinafter, for convenience of description, the endoscope system 1 according to the sixth modification will be referred to as an endoscope system 1F.

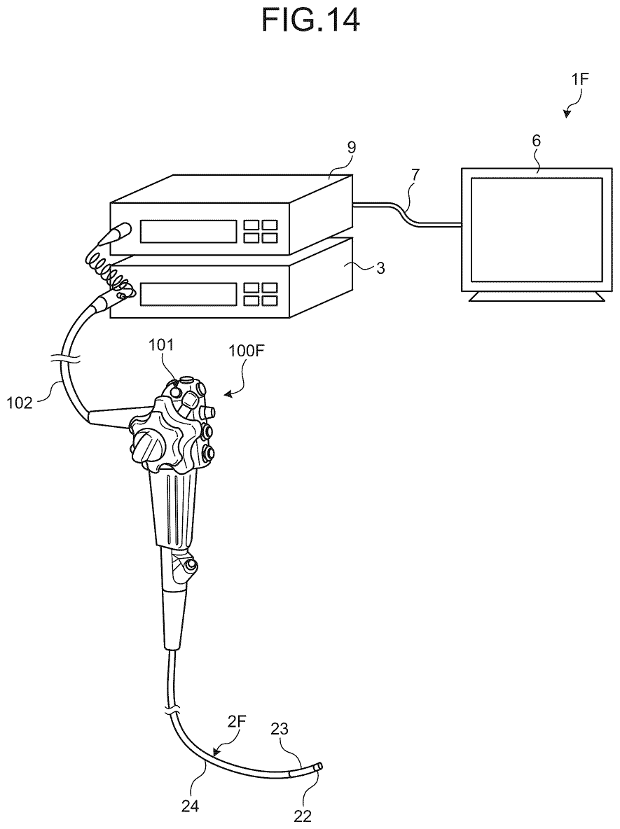

FIG. 14 is a diagram illustrating the sixth modification of the embodiment.

As illustrated in FIG. 14, the endoscope system 1F includes an endoscope 100F that captures an in-vivo image of an observed region by inserting an insertion unit 2F into a living body and outputs a captured image, a light source device 3 that generates illumination light emitted from a distal end of the endoscope 100F, a control device 9 that processes the captured image output from the endoscope 100F, and a display device 6 that is connected to the control device 9 through the second transmission cable 7 and displays an image based on a video signal processed by the control device 9.

In the sixth modification, various endoscopes 100F of different release generations may be connected to the control device 9. More specifically, an existing endoscope 100F for the control device 9 and a later endoscope 100F for the control device 9 may be connected to the control device 9.

As illustrated in FIG. 14, the endoscope 100F includes an insertion unit 2F having a flexible elongated shape, an operating unit 101 connected to a proximal end side of the insertion unit 2F and receiving various operations, and a universal cord 102 extending in a direction different from a direction in which the insertion unit 2F extends from the operating unit 101 and incorporating various cables connected to the light source device 3 and the control device 9.

As illustrated in FIG. 14, the insertion unit 2F includes a distal end portion 22, a bendable bending portion 23 connected to the proximal end side of the distal end portion 22 and configured by a plurality of bending pieces, and an elongated flexible tube portion 24 connected to the proximal end side of the bending portion 23 and having flexibility.

Although not specifically illustrated, a configuration substantially similar to that of the camera head 5 described in the above-described embodiment (the imaging unit 5A1 and the resistor 5A2 in the case of the existing endoscope 100F, and the imaging unit 5B1 and the second endoscope side memory 5B2 in the case of the later endoscope 100F) is incorporated in the distal end portion 22. Then, the captured image captured by the distal end portion 22 (imaging unit) is output to the control device 9 through the operating unit 101 and the universal cord 102.

Even in a case where the configuration of the sixth modification described above is adopted, the same effects as those of the above-described embodiment are obtained.

In the embodiment and the first to sixth modifications described above, the control unit 96 determines whether or not the camera head 5 is the existing camera head 5A or the later camera head 5B, but the present disclosure is not limited thereto, and a difference in the type of the endoscope other than the existing camera head 5A and the later camera head 5B may be determined. In this case, the control unit 96 changes the processing content by the processing module 95 based on the determination result of the type of the endoscope other than the existing camera head 5A and the later camera head 5B.

Note that the following configurations also belong to the technical scope of the present disclosure.
(1) A medical image processing apparatus including: a processing module configured to process image information acquired by an endoscope; and a processor configured to control an operation of the processing module, wherein the processor is configured to change processing contents by the processing module based on a type of the connected endoscope.
(2) The medical image processing apparatus according to (1), further including a memory corresponding to a specific endoscope, the memory being configured to store first setting information related to processing contents of the image information acquired by the specific endoscope by the processing module, wherein the processor is configured to change the processing contents by the processing module to different processing contents depending on whether the endoscope connected to the control device is a first endoscope corresponding to the first setting information stored in the memory or a second endoscope not corresponding to the first setting information.
(3) The medical image processing apparatus according to (2), wherein the second endoscope includes a second endoscope side memory configured to store second setting information regarding processing contents by the processing module of the image information acquired by the second endoscope, and the processor is configured to cause the processing module to execute processing based on the first setting information in a case where the connected endoscope is the first endoscope, and the processor is configured to cause the processing module to execute processing based on the second setting information stored in the second endoscope side memory in a case where the connected endoscope is the second endoscope.
(4) The medical image processing apparatus according to (3), further including an image memory configured to store the image information, wherein the processing module includes: a memory controller including a first register and being configured to control writing of the image information to the image memory and reading of the image information from the image memory based on setting information stored in the first register; and an image processing unit including a second register and being configured to perform image processing on the image information read by the memory controller based on setting information stored in the second register, and the processor is configured to store the first setting information in the first register and the second register in a case where the connected endoscope is the first endoscope, and store the second setting information in the first register and the second register in a case where the connected endoscope is the second endoscope.
(5) The medical image processing apparatus according to (3) or (4), wherein the second endoscope side memory is configured to store second identification information indicative of the second endoscope, and the processor is configured to determine whether the connected endoscope is the first endoscope or the second endoscope based on the second identification information.
(6) The medical image processing apparatus according to (5), wherein the second identification information is included in the second setting information, and the processor is configured to determine whether the connected endoscope is the first endoscope or the second endoscope based on the second identification information included in the second setting information.
(7) The medical image processing apparatus according to (5), wherein the second identification information is stored in the second endoscope side memory as information independent of the second setting information, and the processor is configured to read the second setting information from the second endoscope side memory in a case of determining that the connected endoscope is the second endoscope based on the second identification information.
(8) The medical image processing apparatus according to (3) or (4), wherein the processor is configured to determine whether the connected endoscope is the first endoscope or the second endoscope based on a connector of the connected endoscope.
(9) The medical image processing apparatus according to (3) or (4), wherein the first endoscope includes a first resistor unique to the first endoscope, the medical image processing apparatus includes a second resistor unique to the medical image processing apparatus, and the processor is configured to detect a voltage division ratio based on a resistance value of the first resistor and a resistance value of the second resistor of the connected endoscope, determine that the connected endoscope is the first endoscope in a case where the voltage division ratio is a known value, and determine that the connected endoscope is the second endoscope in a case where the voltage division ratio is not the known value.
(10) The medical image processing apparatus according to any one of (2) to (9), wherein the first endoscope includes a first resistor unique to the first endoscope, the medical image processing apparatus includes a second resistor unique to the medical image processing apparatus, the memory is stored with a voltage division ratio based on a resistance value of each of the first resistors corresponding to a plurality of the first endoscopes and a resistance value of the second resistor in association with the first setting information of each of the plurality of first endoscopes, and the processor is configured to cause the processing module to execute processing based on the first setting information associated with the voltage division ratio based on the resistance value of the first resistor of the first endoscopes and the resistance value of the second resistor in a case where the connected endoscope is the first endoscope.
(11) The medical image processing apparatus according to any one of (2) to (9), wherein the first endoscope includes a first endoscope side memory configured to store first identification information unique to the first endoscope, the memory is stored with each of the first identification information corresponding to a plurality of the first endoscopes in association with the first setting information of each of the plurality of first endoscopes, and the processor is configured to cause the processing module to execute processing based on the first setting information associated with the first identification information of the first endoscopes in a case where the connected endoscope is the first endoscope.
(12) The medical image processing apparatus according to any one of (2) to (9), wherein the first endoscope includes a connector unique to the first endoscope, the memory is stored with connector information indicating each of the connectors corresponding to a plurality of the first endoscopes in association with the first setting information of each of the plurality of first endoscopes, and the processor is configured to cause the processing module to execute processing based on the first setting information associated with the connector information indicating the connectors of the first endoscopes in a case where the connected endoscope is the first endoscope.
(13) A control method executed by a processor of a medical image processing apparatus, the control method including: causing the processor to change a processing content by a processing module configured to process image information acquired by an endoscope based on a type of the endoscope connected to the medical image processing apparatus.

According to the medical image processing apparatus and the control method according to the present disclosure, convenience may be improved.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical image processing apparatus comprising:
a processing module configured to process image information acquired by an endoscope; and
a processor configured to control an operation of the processing module, wherein
the processor is configured to change processing contents by the processing module based on a type of the connected endoscope.

2. The medical image processing apparatus according to claim 1, further comprising a memory corresponding to a specific endoscope, the memory being configured to store first setting information related to processing contents of the image information acquired by the specific endoscope by the processing module, wherein
the processor is configured to change the processing contents by the processing module to different processing contents depending on whether the endoscope connected to the control device is a first endoscope corresponding to the first setting information stored in the memory or a second endoscope not corresponding to the first setting information.

3. The medical image processing apparatus according to claim 2, wherein
the second endoscope includes a second endoscope side memory configured to store second setting information regarding processing contents by the processing module of the image information acquired by the second endoscope, and
the processor is configured to cause the processing module to execute processing based on the first setting information in a case where the connected endoscope is the first endoscope, and
the processor is configured to cause the processing module to execute processing based on the second setting information stored in the second endoscope side memory in a case where the connected endoscope is the second endoscope.

4. The medical image processing apparatus according to claim 3, further comprising an image memory configured to store the image information, wherein
the processing module includes:
a memory controller including a first register and being configured to control writing of the image information to the image memory and reading of the image information from the image memory based on setting information stored in the first register; and
an image processing unit including a second register and being configured to perform image processing on the image information read by the memory controller based on setting information stored in the second register, and
the processor is configured to
store the first setting information in the first register and the second register in a case where the connected endoscope is the first endoscope, and
store the second setting information in the first register and the second register in a case where the connected endoscope is the second endoscope.

5. The medical image processing apparatus according to claim 3, wherein
the second endoscope side memory is configured to store second identification information indicative of the second endoscope, and
the processor is configured to determine whether the connected endoscope is the first endoscope or the second endoscope based on the second identification information.

6. The medical image processing apparatus according to claim 5, wherein
the second identification information is included in the second setting information, and
the processor is configured to determine whether the connected endoscope is the first endoscope or the second endoscope based on the second identification information included in the second setting information.

7. The medical image processing apparatus according to claim 5, wherein
the second identification information is stored in the second endoscope side memory as information independent of the second setting information, and
the processor is configured to read the second setting information from the second endoscope side memory in a case of determining that the connected endoscope is the second endoscope based on the second identification information.

8. The medical image processing apparatus according to claim 3, wherein
the processor is configured to determine whether the connected endoscope is the first endoscope or the second endoscope based on a connector of the connected endoscope.

9. The medical image processing apparatus according to claim 3, wherein
the first endoscope includes a first resistor unique to the first endoscope,
the medical image processing apparatus includes a second resistor unique to the medical image processing apparatus, and
the processor is configured to
detect a voltage division ratio based on a resistance value of the first resistor and a resistance value of the second resistor of the connected endoscope,
determine that the connected endoscope is the first endoscope in a case where the voltage division ratio is a known value, and
determine that the connected endoscope is the second endoscope in a case where the voltage division ratio is not the known value.

10. The medical image processing apparatus according to claim 2, wherein
the first endoscope includes a first resistor unique to the first endoscope,
the medical image processing apparatus includes a second resistor unique to the medical image processing apparatus,
the memory is stored with a voltage division ratio based on a resistance value of each of the first resistors corresponding to a plurality of the first endoscopes and a resistance value of the second resistor in association with the first setting information of each of the plurality of first endoscopes, and
the processor is configured to cause the processing module to execute processing based on the first setting information associated with the voltage division ratio based on the resistance value of the first resistor of the first endoscopes and the resistance value of the second resistor in a case where the connected endoscope is the first endoscope.

11. The medical image processing apparatus according to claim 2, wherein
the first endoscope includes a first endoscope side memory configured to store first identification information unique to the first endoscope,
the memory is stored with each of the first identification information corresponding to a plurality of the first endoscopes in association with the first setting information of each of the plurality of first endoscopes, and
the processor is configured to cause the processing module to execute processing based on the first setting information associated with the first identification information of the first endoscopes in a case where the connected endoscope is the first endoscope.

12. The medical image processing apparatus according to claim 2, wherein
the first endoscope includes a connector unique to the first endoscope,
the memory is stored with connector information indicating each of the connectors corresponding to a plurality of the first endoscopes in association with the first setting information of each of the plurality of first endoscopes, and
the processor is configured to cause the processing module to execute processing based on the first setting information associated with the connector information indicating the connectors of the first endoscopes in a case where the connected endoscope is the first endoscope.

13. A control method executed by a processor of a medical image processing apparatus, the control method comprising:
causing the processor to change a processing content by a processing module configured to process image information acquired by an endoscope based on a type of the endoscope connected to the medical image processing apparatus.
